(19) United States
(12) Patent Application Publication
(10) Pub. No.: US 2014/0220568 A1
(43) Pub. Date: Aug. 7, 2014

(54) MEANS AND METHODS FOR THE DETERMINATION OF PREDICTION MODELS ASSOCIATED WITH A PHENOTYPE

(71) Applicants: Dirk G. Inze, Moorsel-Aalst (BE); Nathalie Gonzalez, Merelbeke (BE); Stefanie De Bodt, Gent (BE); Yvan Saeys, Oudenaarde (GE)

(72) Inventors: Dirk G. Inze, Moorsel-Aalst (BE); Nathalie Gonzalez, Merelbeke (BE); Stefanie De Bodt, Gent (BE); Yvan Saeys, Oudenaarde (GE)

(73) Assignees: Universiteit Gent, Gent (BE); VIB VZW, Gent (BE)

(21) Appl. No.: 14/129,266

(22) PCT Filed: Jun. 25, 2012
(86) PCT No.: PCT/EP2012/062234

§ 371 Date: Feb. 12, 2014

Related U.S. Application Data
(60) Provisional application No. 61/571,302, filed on Jun. 24, 2011.

(30) Foreign Application Priority Data
Jun. 28, 2011 (GB) 1110888.3

(51) Int. Cl. C12Q 1/68

(52) U.S. Cl. CPC C12Q 1/6869
USPC 435/6.11

(57) ABSTRACT

The disclosure provides methods and means for identifying plants comprising a plant phenotype of interest. In particular, the disclosure provides breeding tools that can be used for the selection of a plant comprising a phenotype of interest and for the selection of an optimal plant genotype for the introduction of a trait.

### CROSS-REFERENCE TO RELATED APPLICATIONS

This application is a national phase entry under 35 U.S.C. §371 of International Patent Application PCT/EP2012/062234, filed Jun. 25, 2012, designating the United States of America and published in English as International Patent Publication WO 2012/175736 A1 on Dec. 27, 2012, which claims the benefit under Article 8 of the Patent Cooperation Treaty and under 35 U.S.C. §119(e) to U.S. Provisional Patent Application Ser. No. 61/571,302, filed Jun. 24, 2011, and to United Kingdom Patent Application Serial No. 1110888.3, filed Jun. 28, 2011.

### TECHNICAL FIELD

The present disclosure relates to the field of plant molecular biology. More particularly, the disclosure relates to a method for selecting a plant with a predicted phenotype of interest. The disclosure further relates to a method for the selection of an optimal plant genotype for the introduction of one or more transgenes. As such, the disclosure offers methods for breeding decisions for the selection of a plant based on predicting the presence of a plant phenotype in a particular plant and selecting the plant for subsequent breeding.

### BACKGROUND

The heritable differences in genomes that are reflected in the variation of the expression of a particular phenotype, and which contribute to the range of phenotypes observed for any of a number of phenotypes, form the basis for decisions in plant and animal breeding. Typically, any one phenotype will be modulated by multiple genetic factors and differences of these genetic factors between individuals can be associated with a variation in the phenotypic outcome between individuals. In the instance where the phenotype is the product of one or more transgenes or where the phenotype is influenced by one or more transgenes, it is expected that several genetic factors in the organism's genome contributes to the phenotype of the transgene or to the phenotype influenced by the transgene. The possibility to manipulate plant phenotypes that affect the production of food, fiber and renewable energy has important agricultural consequences. Indeed, the most important goal in plant breeding is to meet a product concept by selecting the most promising plants as founders for further breeding or by selecting the best germplasm candidates for introduction of a transgene. Breeders are faced with a constant challenge to improve and shorten the timelines of the breeding processes. The outcome of a phenotype may be impacted by constitutive genes or more typically by genes which are only expressed at specific points in time during development in a plant. Allelic variants of constitutive genes, copy number variations, deletions, the presence of specific microRNA populations, promoter variations may all impact the genetic outcome of a particular phenotype. There is currently no magic approach for identifying genes which are correlated with important plant phenotypes. Forward genetics is limited as mutations in many genes may generate only moderate or weak phenotypes. Similarly, although reverse genetics allows for directed assay of gene perturbations, saturated phenotyping for many plant phenotypes is impractical.

In the prior art, several attempts were made to identify simple genes, like individual transcripts, in order to describe certain plant phenotypes, and even more complex plant phenotypes like biomass production and growth. Most of these attempts had no satisfactory outcome, since complex traits usually are related to a more complex network of transcripts all partially representing such complex phenotypes.

Another approach which has been proposed in the art is the computational identification of likely candidate genes for desired phenotypes, allowing for focused, efficient use of reverse genetics. An emerging approach for prioritizing candidate genes is network-guided guilt by association. In this approach, functional associations are first determined between genes in a genome on the basis of extensive experimental data sets such as microarray data sets. Probabilistic functional gene networks aim at integrating heterogeneous biological data into a single model, enhancing both model accuracy and coverage. Once a suitable network is generated, new candidate genes are proposed for phenotypes based upon network associations with genes previously linked to these phenotypes. Such network-guided screening has been successfully applied to the reference flowering plant, Arabidopsis thaliana (Insuk Lee et al., (2009) Nature Biotechnology 28(2) 149). Obviously, a key to progress towards breeding better crops has been to understand the changes in cellular, biochemical and molecular machinery that occur associated with a particular phenotype. The development of genetically engineered plants by the overexpression or downregulation of selected genes seems to be a viable option to hasten the breeding of “improved” plants but has thus far not generated a significant impact on the generation of crops with improved quantitative traits such as yield, drought tolerance and abiotic stress tolerance.

A further aspect is the unpredictable performance of a particular transgene in a given plant genetic background. In the past, a great deal of scientific effort has been invested in the development of transformation systems in plants. Transformation is noiinally used to introduce single novel genes into a plant and this gene usually modifies a single important characteristic of the recipient line. There are still barriers, however, to the transformation of agronomically-proven important crop genotypes, and several of these can be overcome by conventional crossing strategies. In some crop species only certain cultivars can be transformed efficiently and these often yield less than the most modern varieties and elite breeding material. In these cases, conventional breeding is used to transfer a promising transgene from a donor cultivar to a modern variety, and thus combine benefits of transformation and conventional breeding methods. To have the optimum potential, transgenic varieties should have genetic backgrounds which have been selected for maximum yield and good quality characteristics under normal agronomic conditions. The genotype of an elite variety is a complex assembly of genes controlling a large number of characters. To have the best effect, transgenes should be introduced (e.g., by crossing or transformation) in genetic backgrounds with an optimal plant transcriptional network able to synergize with the introduced transgene. It is known that every genetic background has its modifiers genes which influence the expression of a particular transgene. The speed with which transgenes are transferred into improved genetic backgrounds is accelerated by the application of marker-assisted breeding techniques. Marker-assisted backcrossing programs can introgress transgenes into elite varieties by selecting indirectly for the large numbers of alleles (with complex interactions) that make up a superior genotype. The latter is done without the need to identify the individual genes involved or to understand their modes of action. In the prior art methods have been described for the identification of loci modulating transgene performance in plant breeding through the screening of germplasm entries (see, for example, WO2009002924).

Notwithstanding the foregoing, the current scientific opinion is that distinct gene networks operate in different genetic backgrounds or exist in plants grown in various environmental conditions. These gene networks contribute to the presence of a particular phenotype. A specific gene network for a given phenotype could be a valuable breeder tool to assist breeders in selecting the most valuable plant, with an expected phenotype, from, for example, a germplasm collection of immature plants or could assist breeders in selecting the most valuable genotype for the introduction of a trait able to influence a particular phenotype. It is a challenge to identify such gene networks which are specifically associated with a predicted phenotype of interest in a plant.

### BRIEF SUMMARY

Demonstrated is that a combination of a set of absolute expression-values of specific genes in combination with a statistical model (i.e., herein defined as a plant phenotype predictor) is associated with a high likelihood of a specific predicted phenotype of interest. In other words, it was found that the specific composition and its absolute expression values of a gene expression network represents (or is associated with or corresponds with) a complex phenotype of interest of a plant, such as, for example, leaf biomass production.

Accordingly, the disclosure relates to methods of predicting a future phenotype of interest in an organism such as a plant. In one embodiment, the disclosure enables the artisan to associate the presence of absolute gene expression signatures in plants, in combination with a suitable statistical model, with a predicted phenotype of interest in an organism such as a plant.

Accordingly, the present disclosure for the first time provides the above-described direct proof that the output of a specific plant phenotype predictor is highly correlated with the expression of a certain phenotype of a plant, like, for example, leaf biomass production. One further merit of the disclosure is the successful demonstration that a future plant phenotype can be predicted based on the presence of an absolute gene expression signature in a plant present in a collection of immature plants.

Moreover, it could be shown in the context of this disclosure, that for training the statistical model to be applied for predicting the phenotype of interest, not necessarily those plants have (or this group of plants has) to be analyzed (e.g., by performing a gene expression profile analysis of a particular tissue of each of the plants) for which the prediction is intended to be carried out. As also exemplary, shown in the appended experimental part, the prediction of the expression of a phenotype can also be carried out for plants, which were not employed for establishing the plant phenotype predictor. The latter means that the plant phenotype predictor was calculated (or established) in a training population and that the plant phenotype predictor can be used in other plants, which do not belong to the training population. In still other words, a prediction of the presence of a future phenotype is also possible for such plants which were cultivated independently from those plants which were initially employed (or “analyzed,” according to the methods described herein) for the training of the correlation model. Hence, the methods provided herein can also be applied, when the (group of) plants employed for generating the correlation model were grown independently of the (group of) plants for which the phenotype of interest is to be predicted. The meaning of plants which “were employed” refers to the fact that a gene expression profiling method is applied on the plants. It is expected that slight differences in environmental conditions, which exist between independent cultivations, do not constrain the predictiveness of the plant phenotype predictor with respect to the potential for the presence of a corresponding plant phenotype. These are further advantages of the present disclosure.

In still other words, the present disclosure, relates in a genotype independent manner to the identification of plants comprising a predicted phenotype of interest based on calculating the correspondence between a plant phenotype predictor and the phenotype of interest with a statistical model.

The findings provided herein offer agricultural potential for a number of applied purposes. For example, the possibility to predict the presence of certain plant phenotypes on the basis of the presence of one or more absolute gene expression signatures, in combination with an established statistical model established in a training set of plants, in one or more immature plants present in a group of plants revolutionizes the selection and thus breeding processes of plants. Particularly, with respect to biomass producers, such as trees that are cultivated for many years or even decades before harvest, the means and methods of the present disclosure are highly advantageous. The identification of certain plants that are capable of expressing (a) certain phenotype(s) in a desired manner, for example, potentially high biomass producers, already at an early growth stage, preferably an immature growth stage, even at the seed stage, can result in enormous time and cost-savings, especially in selection and breeding procedures.

### BRIEF DESCRIPTION OF THE DRAWINGS

FIG. 1: Correlation initial leaf size versus final leaf size.

FIG. 2a: Prediction of final leaf size. Classification results using support vector machines on 100 real (dark) and random (grey) datasets.

FIG. 2b: Prediction of leaf size at harvest. Classification results using support vector machines on 100 real (dark) and random (grey) datasets.

FIG. 2c: Prediction of final rosette size. Classification results using support vector machines on 100 real (black) and random (grey) datasets.

FIG. 2d: Classification based on mechanism results using support vector machines on 100 real (black) and random (grey) datasets.

FIG. 3: Summary of regression analysis

FIG. 4: Co-expression network of the growth predictors based on the expression data in small plants (PCC >0.65).

FIG. 5: Co-expression network of the growth predictors based on the expression data in large plants (PCC >0.65).

### DETAILED DESCRIPTION

The definitions and methods provided, define the present disclosure and guide those of ordinary skill in the art in the practice of the present disclosure. Unless otherwise noted, terms are to be understood according to conventional usage by those of ordinary skill in the relevant art. Definitions of common terms in molecular biology may also be found in Alberts et al., Molecular Biology of The Cell, 5th Edition, Garland Science Publishing, Inc.-New York, 2007; Rieger et al., Glossary of Genetics: Classical and Molecular, 5th edition, Springer-Verlag: New York, 1991; King et al., A Dictionary of Genetics, 6th ed, Oxford University Press: New York, 2002; and Lewin, Genes IX, Oxford University Press: New York, 2007. The nomenclature for DNA bases as set forth at 37 CFR §1.822 is used. To facilitate the understanding of this disclosure a number of terms are defined below. Terms defined herein (unless otherwise specified), have meanings as commonly understood by a person of ordinary skill in the areas relevant to the present disclosure. As used in this specification and its appended claims, terms such as “a,” “an” and “the” are not intended to refer to only a singular entity, but include the general class of which a specific example may be used for illustration, unless the context dictates otherwise. The terminology herein is used to describe specific embodiments of the disclosure, but their usage does not delimit the disclosure, except as outlined in the claims.

An “allele” refers to an alternative sequence at a particular locus, the length of an allele can be as small as 1 nucleotide base, but is typically larger. Allelic sequence can be denoted as nucleic acid sequence or as amino acid sequence that is encoded by the nucleic acid sequence. A “locus” is a position on a genomic sequence that is usually found by a point of reference, e.g., a short DNA sequence that is a gene or part of a gene or intergenic region. A locus may refer to a nucleotide position at a reference point on a chromosome, such as a position from the end of the chromosome. The ordered list of loci known for a particular genome is called a genetic map. A variant of the DNA sequence at a given locus is called an allele and variation at a locus, i.e., two or more alleles, constitutes a polymorphism. The polymorphic sites of any nucleic acid sequence can be determined by comparing the nucleic acid sequences at one or more loci in two or more germplasm entries. “Polymorphism” means the presence of one or more variations of a nucleic acid sequence at one or more loci in a population of one or more individuals. The variation may comprise, but is not limited to, one or more base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The disclosure can be commonly found, or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a haplotype, a RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, and a methylation pattern may comprise polymorphisms. In addition, the presence, absence, or variation in copy number of the preceding may comprise a polymorphism. As used herein, “genotype” means the genetic component of the phenotype and it can be indirectly characterized using markers or directly characterized by nucleic acid sequencing or more specifically, in the context of the present disclosure, by the association with one or more plant phenotype predictors. As used herein “phenotype” means the detectable characteristics of a cell or organism which can be influenced by gene expression. The term “transgene” means nucleic acid molecules in the form of DNA, such as cDNA or genomic DNA, and RNA, such as mRNA or microRNA, which may be single or double stranded. The term “event” refers to a particular transformant comprising a transgene. In a typical transgenic breeding program, a transformation construct responsible for a trait is introduced into the genome via a transformation method. Numerous independent transformants (events) are usually generated for each construct. These events are evaluated to select those with superior performance.

The term “inbred” means a line that has been bred for genetic homogeneity. Without limitation, examples of breeding methods to derive inbreds include pedigree breeding, recurrent selection, single-seed descent, backcrossing, and doubled haploids. The term “hybrid” means a progeny of mating between at least two genetically dissimilar parents. Without limitation, examples of mating schemes include single crosses, modified single cross, double modified single cross, three-way cross, modified three-way cross, and double cross, wherein at least one parent in a modified cross is the progeny of a cross between sister lines. “Germplasm” includes breeding germplasm, breeding populations, collection of elite inbred lines, populations of random mating individuals, and bi-parental crosses.

In one embodiment, the disclosure provides a method for predicting the presence of a plant phenotype in plants comprising the steps of: a) determining the presence of a plant phenotype in individuals of a group of plants, wherein the individual plants display a variation of the phenotype, and wherein the group of plants faun a training population b) isolating a specific tissue from each plant of the group of plants, c) carrying out an expression profile analysis on the tissues, d) select a number of absolute gene expression value signatures present in the gene expression profile analysis, e) build statistical models (either through regression or classification models) using these signatures to predict the presence of a plant phenotype, and f) determine the prediction quality using a cross-validation setup, thereby employing “correlation” as a measure for the quality of the regression models, and accuracy as a measure for the quality of the classification models and thereby obtaining a plant phenotype predictor and g) using the plant phenotype predictor obtained in step f) for predicting the plant phenotype in a plant, which was not used in the training population of step a).

In a particular embodiment, the method for predicting the presence of plant phenotypes in plants comprises the isolation of specific tissues from immature plants present in the group of plants (step b) of the previous embodiment).

In another embodiment, the disclosure provides a method for identifying a plant phenotype predictor, which is correlated with the presence of a predicted plant phenotype of interest comprising the steps of: a) providing a collection of (immature) plants displaying an expected variation of the phenotype of interest, b) isolating a specific tissue from each (immature) plant of the collection of plants, c) carrying out an expression profile analysis on the tissues, d) select a number of absolute gene expression value signatures present in the gene expression analysis, e) build statistical models (either through regression or classification models) using these signatures to predict the presence of a plant phenotype, and f) determine the prediction quality using a cross-validation setup, thereby employing “correlation” as a measure for the quality of the regression models, and accuracy as a measure for the quality of the classification models and g) identifying a plant phenotype predictor, which is correlated with the presence of a predicted plant phenotype of interest.

In yet another embodiment, the disclosure provides a method for producing a plant comprising a predicted plant phenotype of interest comprising the steps of: a) determining the presence of a plant phenotype in individuals of a group of plants, wherein the individual plants display a variation of the phenotype, and wherein the group of plants faun a training population b) isolating a specific tissue from each (immature) plant of the group of plants, c) carrying out an expression profile analysis on the tissues, d) select a number of absolute gene expression value signatures present in the gene expression profile analysis, e) build statistical models (either through regression or classification models) using these signatures to predict the presence of a plant phenotype, and f) determine the prediction quality using a cross-validation setup, thereby employing “correlation” as a measure for the quality of the regression models, and accuracy as a measure for the quality of the classification models and thereby obtaining a plant phenotype predictor and g) using the plant phenotype predictor obtained in step f) for predicting the plant phenotype in a plant, which was not used in the training population of step a).

In a preferred embodiment, the “specific tissue” is determinative for the predicted phenotype of interest. For example, the ear meristem is isolated if the phenotype of interest is (enhanced) ear development. In yet another example, leaf meristem is isolated if the phenotype of interest is leaf development.

In a particular embodiment, a collection of immature plants is a reference collection (also designated as a “training collection”) of mature or immature plants. A reference collection preferably consists of plants derived from the same genus, more preferably from the same species. Typically, a reference collection is a collection of plant ecotypes or a germplasm collection of plants derived from the same species. A reference collection can be, for example, a collection of canola, corn or rice plants but can also consist of model plants, such as, for example, Arabidopsis thaliana or Brachypodium distachyon. A reference collection can also form a collection of plants, which have been subjected to different environmental conditions, such as cold stress, heat stress, biotic stress, drought stress, UV-stress and the like. A reference collection can also consist of a collection of plants each comprising at least one transgene or a collection of plants each comprising at least one different transgene. In a preferred embodiment, a transgene encodes for a transgenic trait and the transgenic trait has an effect on the (predicted) phenotype of interest. The effect of a transgenic trait on a (predicted) phenotype of interest means that the transgenic trait is preferably able to enhance the phenotypic expression of interest or, less preferably, to reduce the phenotypic expression of interest.

Typically, a trait is, in the context of the present disclosure, an exogenously added characteristic encoding a phenotype, which can be introgressed through classical breeding (i.e., crossing and selection) or through recombinant transformation. A trait can be a transgenic trait or a native trait.

Typically, a native trait is a naturally occurring recognized non-transgenic plant phenotype, which is heritable and can be used in several varieties of at least one plant species. Alternatively, a native trait is man-made and can be generated through mutagenesis of plants. A native trait is often introgressed in a variety or plant species of choice by breeding. Introgression of a native trait can be carried out with the aid of molecular markers flanking the locus or loci comprising the trait of interest. Non-limiting examples of native traits, which can be used are emergence vigor, vegetative vigor, disease resistance, branching, pre-mature sprouting, bolting, flowering, seed set, seed size, seed density, etc.

Typically, a transgenic trait is used where the expression levels, location or timing of the expression of a gene product is usefully altered, or for a gene derived from a species, which cannot be crossed with the organism, wherein the transgenic trait needs to be introgressed.

Non-limiting examples of transgenic traits, which can be used in accordance with the present disclosure, are traits offering intrinsic yield production, abiotic stress tolerance (including heat, drought and cold), nitrogen efficiency, disease resistance, insect resistance, enhanced amino acid content, enhanced protein content, modified fatty acids, enhanced starch production, phytic acid reduction, enhanced nutrition, improved processing trait and improved digestibility.

The wording “a tissue, which is determinative for the phenotype of interest” means that the phenotype is not visible present in the tissue, isolated from the immature plant, but that the phenotype of interest is only displayed when the plant is grown to maturity. In other words, the tissue derived from the immature plant is deteiminative for a predicted phenotype present in the mature plant, the predicted phenotype being statistically associated with a plant phenotype predictor, which is calculated with a statistical model based on the absolute expression values of genes present in a plant transcriptional profile derived from a specific tissue. A tissue, in the context of the present disclosure, can, for instance, be fresh material such as a tissue explant, which may be directly subjected to nucleic acid extraction such as RNA extraction. Plant tissues may also be stored for a certain time period, preferably in a fonii that prevents degradation of the nucleic acids in the tissue sample. A tissue sample may be frozen in, for instance, liquid nitrogen or may be lyophilized. Tissue samples may be prepared according to methods known to the person skilled in the art and should be carried out in a way suitable to the respective method of the present disclosure to be applied. Care should be taken that the nucleic acids to be analyzed are not degraded during the extraction process. It is preferred that a step for obtaining the tissue of the immature plant, for which the plant expression signature is to be determined in the context of the present disclosure, is as little invasive as possible for the plant. The latter means that the plants to be tested are disturbed as little as possible in their development, when applying the methods of the disclosure. The latter is particularly relevant for those methods, disclosed herein, that refer to the prediction of the expression of a plant phenotype of interest or the selection of a plant (genotype) of interest. Such methods are, for example, the methods for breeding of a plant, as further disclosed herein. Accordingly, the plant tissue is preferably of such part or organ of a plant, which is not crucial for the development of the plant. Non-limiting examples for such a part or organ may be a leaf (e.g., the third leaf in development, a cotyledon), a bud, a root meristem, an ear meristem, an intercalary meristem and the like.

In a specific embodiment, a plant phenotype predictor (which is correlated with the expression of a plant phenotype or with the expression of an expected plant phenotype) can be used to determine the potential for the expression of a plant phenotype in a collection of plants. The meaning of the term “potential for the expression of a plant phenotype” refers to a status of a plant at a certain growth stage in time that determines a future expression of a plant phenotype, i.e., an expression of the plant phenotype after the certain growth stage in time. Preferably the “growth stage in time” of the plant is a growth stage present in an immature plant. In still other words, the “potential for the expression” means the potential (or capacity) for the expression in the future (e.g., the mature plant).

In another embodiment, the disclosure provides a method for selecting a plant comprising a phenotype of interest comprising the following steps: a) providing a collection of immature plants displaying a variation of a phenotype of interest wherein the phenotype is only visible when the plants are mature, b) isolating a tissue from each immature plant in the collection wherein the tissue is determinative for the phenotype, c) carrying out a transcriptional profile on each of the tissues, d) evaluating the correlation between a plant phenotype predictor present in the transcriptional profile and the plant phenotype of interest, the correlation being previously measured by i) providing a reference collection of immature plants displaying an expected variation of the phenotype of interest, ii) isolating a tissue from each of the plants present in the reference collection, iii) carrying out a transcriptional profile on each of the tissues, and iv) determining, with a statistical model, a plant phenotype predictor present in the transcriptional profile, which is associated with the phenotype, and e) based on the evaluation in step d) selecting a plant comprising a phenotype of interest.

In a particular embodiment, the plant phenotype predictor comprises the expression levels of less than 200 genes, less than 150 genes, less than 100 genes, less than 75 genes, less than 50 genes, less than 40 genes, less than 30 genes, less than 25 genes or even less than 20 genes.

In another particular embodiment, the plant phenotype predictor comprises the expression levels of between 100 and 200 genes. In another particular embodiment, the plant phenotype predictor comprises the expression levels of between 100 and 150 genes. In another particular embodiment, the plant phenotype predictor comprises the expression levels of between 50 and 100 genes. In yet another particular embodiment, the plant phenotype predictor comprises the expression levels of between 25 and 50 genes. In yet another embodiment, the plant phenotype predictor comprises the expression levels of between 10 and 25 genes. In yet another embodiment, the plant phenotype predictor comprises the expression levels of between 5 and 10 genes. In yet another embodiment, the plant phenotype predictor comprises the expression levels of between 2 and 5 genes. Examples of plant phenotype predictors are mentioned in the examples section, such as in Table 5.

In a particular embodiment, the methods for selection of a plant comprising a phenotype of interest, herein described further, comprise the use of the selected plant for a breeding activity and the production of a progeny (i.e., seeds and plants) of the breeding activity.

In a particular embodiment, the selected plant is a particular germplasm entry and the germplasm entry is used in making a breeding cross.

In another particular embodiment, the selected plant is a germplasm entry and the selected germplasm entry is used as a donor to introgress a genomic region into at least one recipient germplasm entry.

The term “the expression level of a gene” means here the absolute amount of the abundance of the mRNA of a gene in a particular plant, plant tissue or in a group of pooled plants of the same genotype, wherein the plants have grown in the same conditions.

In another embodiment, a plant tissue derived from an immature plant can be any tissue derived from an immature plant provided the tissue is determinative for the future phenotype and the phenotype is not yet visibly present in the tissue. Typical tissues are derived from roots, cotyledons and leaves. In a specific embodiment, a tissue is a tissue responsible for the division of new cells such as a meristematic tissue. Typical meristems are apical meristems, lateral meristems and intercalary meristems.

A “plant phenotype of interest” may, in the context of the present disclosure, for example, be of morphological nature, anatomical nature, physiological nature, eco-physiological nature, patho-physiological nature, and/or ecological nature, and the like.

For example, “plant phenotypes” of morphological nature may be size, weight, number, surface area, and the like, of roots (like, e.g., storing roots), of shoots, like side shoots (like e.g., storing shoots), of leaves (like e.g., (succulent) storing leaves), of flowers or inflorescences, of fruits, of seeds (like, e.g., grains), and the like. Other examples of “phenotypes” of morphological nature may be size, height, weight, and the like, of the whole plant.

“Plant phenotypes” of anatomical nature, for example, may be the anatomical structure of vascular bundles (like, for example, development of the crown syndrome), of the medulla, of the wood or of other tissues, and the like.

“Plant phenotypes” of physiological nature, for example, may be contents of compounds, in particular storage compounds, like lignin, cellulose, starch or sugars (or other nutrients like fats or proteins), fibers, water, vitamins or compounds of the secondary metabolism of plants, fertility, and the like.

“Plant phenotypes” of eco-physiological nature, for example, may be tolerance or resistance against environmental influences (including “man-made” environmental influences) like drought, heat, cold, hypoxia and/or heavy metals and the like.

“Plant phenotypes” of pathophysiological nature, for example, may be tolerance or resistance against pathogens like viruses, fungi, bacteria and/or nematodes, and the like.

“Plant phenotypes” of ecological nature, for example, may be the potential for attraction or repellence to phyto-phages or nectar/pollen-collecting animals (like insects), the capacity to adapt to changes in the environment, and the like.

It is of particular note that a given “plant phenotype,” in the context of the present disclosure, may not belong to only a single one of the above mentioned categories, but also to several of them, and, furthermore, to other categories not explicitly mentioned herein.

The herein mentioned categories of plant phenotypes, as well as the herein mentioned examples of plant phenotypes, are by far not limiting. Further “plant phenotypes” of plants, e.g., in the form of detectable features or characters, are well known in the art. The person skilled in the art is readily in the position to figure out further “plant phenotypes,” particularly, of plant phenotypes, the observation of which is economically desired, based on his common general knowledge and the disclosure in the prior art. The above mentioned and also further “plant phenotypes” being observable, in the context of the present disclosure, can particularly be deduced from corresponding pertinent literature.

Another particular example of a “plant phenotypes,” which expression may be predicted or determined, in accordance with this disclosure, is the area of leaves of a plant. In the appended examples, it is, inter alia, shown that the expression of this plant phenotype can be predicted/determined on in accordance with the methods of this disclosure.

In the context of the present disclosure, the term “comprising a phenotype of interest” can also be construed as “expressing (or “displaying,” which is equivalent) a phenotype of interest” and the wordings refer to how a phenotype is expressed in terms of measurable parameters. For example, in case the “phenotype” to be observed is biomass production or, for example, growth or, for example, leaf area, the parameters, for example, are volume/mass expansion per time or volume/mass at a certain point in time. In this context, “mass” can mean dry weight or fresh weight of (a) plant(s) to be employed. Further non-limiting examples of measurable parameters in this context are number, amount, concentration, length, density, area, flexibility and the like.

In the present disclosure, a “plant phenotype predictor” consists of the absolute expression values of a chosen set of genes present in a transcriptional profile (e.g., a transcription profile obtained from an immature plant tissue or a particular plant tissue), which in combination with a statistical model, is able to predict the phenotype of interest in plants, which were not used for the identification of the plant phenotype predictor.

In the methods for determining a plant phenotype predictor, as herein described before a reference collection of plants can be employed, which differ in their (potential for) expression of the (future) phenotype.

In a particular embodiment, a reference collection is a collection of immature plants. The tend “immature plants that differ in their potential for expression of a future phenotype of interest,” as used herein, means that different individual plants of a group of plants, as defined herein, exhibit different (potentials for) expression of a future phenotype. Particularly, this means that the potential for expression of a phenotype of interest of a group of plants is reduced or enhanced compared to a certain standard, like, for example, the potential for the expression of the phenotype of interest of at least one other plant of the group of plants or the averaged potential for the expression of the phenotype of a certain number of plants of the group of plants. For example, the individuals of an A. thaliana RIL population can exhibit a range of different presence of a particular phenotype in plant phenotypes (e.g., leaf growth production) among each other, following a relatively equal distribution. Such an A. thaliana RIL population and of their test crosses is a non-limiting example for a group of plants, which can be employed in the context of the present disclosure to establish the correlation between a plant phenotype predictor and a phenotype of interest.

In one embodiment, it is possible that the potential for the presence of a (future) phenotype of interest to be observed of the different plants of a group of plants to be employed herein exhibit a wide range and/or show a relatively equal distribution within this range. Without being bound by theory, such a wide range and/or equal distribution may result in particularly reliable outcomes of the analyses of the predictive quality between a plant phenotype predictor and the potential for the presence of a future phenotype, as disclosed herein.

Advantageously, in the mature plants an expression that can be detected of a plant phenotype to be observed herein, may, for example, be visually identifiable, such as a morphological (or anatomical) outcome. However, such expression of a plant phenotype of interest may, for example, also be non-visually identifiable, such as a physiological outcome, like an outcome of the chemical composition of certain compartments of a plant or a plant cell (like, e.g., cell wall, cytosol, membrane systems (like the endoplasmic reticulum) or lumens enclosed therein (like the intrathylacoid lumen or the grana matrix of chloroplasts), and the like.

It is clear that the “potential for the presence (or the expression) of a future phenotype in a plant” may be influenced by environmental factors. For example, such factors are light supply, light quality, water supply, nitrogen supply, soil composition, biotic stresses and abiotic stresses such as drought, heat, salt and the like.

Thus, the “presence of a future phenotype” on the one hand may be a function of, i.e., determined by, the genetic background of a phenotype (the absolute expression of a set of gene(s) that determine the phenotype of interest), and on the other hand a function of the possible environmental impact on the absolute expression values of the genes, and hence on the presence of the plant phenotype. Accordingly, without being bound by theory, a plant phenotype predictor that represents a certain (potential for) presence of a plant phenotype in a plant, selected from a collection of plants, may reflect both the specific genetic background of the plants and the environmental impact on (the potential for) the presence of the phenotype, as well as the interaction of these two factors.

In a preferred embodiment of the present disclosure, it is particularly desired for the herein provided methods of the present disclosure, that the (potential for) expression of a future phenotype that differs between plants to be tested/observed, reflects differences in the genetic background of the plants.

A “gene expression profile” includes, but is not limited to, gene expression profiles as generally understood in the art. A gene expression profile of a number of genes in a plant tissue (e.g., leaf, meristem or seed) derived from a specific plant, typically contains a number of genes differentially expressed in comparison to the average expression of the genes in the pool of a genetically diverse population of plants. A gene that appears in a gene expression profile, whether by up-regulation or down-regulation is said to be a member of the gene expression profile. It is understood that such a gene expression profile can be refined by, for example, measuring the co-expression of the differentially expressed genes in one or more several expression networks. A gene expression profile of a group of genes, typically consists of a set of absolute expression values of the group of genes. Hence, by selecting different genes derived from the group of genes it is possible—in combination with a statistical model that predicts the phenotype of interest—it is possible to obtain alternative plant phenotype predictors. The skilled person will generally choose the determined plant phenotype predictor which has the highest predictive value. Examples of refinements of gene expression profiles through the identification of a plant phenotype predictor associated with a plant phenotype of interest, is presented in the example section. In a particular embodiment, the constituents to determine a plant phenotype predictor, in combination with a statistical model, are a set of absolute expression values of genes, which encode, for example, transcription factors. In yet another particular embodiment, the constituents of such a plant phenotype predictor are genes encoding signal transduction molecules such as kinases, phosphatases GTP-binding proteins and the like. In yet another embodiment, the constituents of a plant phenotype predictor are transcription factors, signal transduction molecules and histon acetyltransferases.

While not intending to limit the disclosure to a particular explanation of the predictive quality between a plant phenotype predictor with a phenotype of interest in a plant tissue derived from an immature plant, wherein the tissue is determinative for the future phenotype, it is thought that certain expression values of genes, forming part of a specific plant phenotype predictor, are only active in a specific tissue in immature plants while the same genes are not necessarily active at the mature stage of the plant, (i.e., when the phenotype is present).

Several methods for determining the expression level of a gene (or genes) are known in the art. A gene expression profile may be “determined,” without limitation, by means of DNA microarray analysis, PCR, quantitative RT-PCR, RNA-sequencing, etc. These are referred to herein collectively as “nucleic-acid based determinations” or “assays.” Alternatively, methods as multiplexed immunofluorescence microscopy or flow cytometry may be used. Plant phenotype predictors, present in gene expression profiles, may be also conveniently determined, in a particularly preferred approach, with RNA-seq or the nCounter Nanostring technology (see the examples section).

The aforementioned methods for examining gene sets employ a number of well-known methods in molecular biology, to which references are made herein. A gene is a heritable chemical code resident in, for example, a cell, virus, or bacteriophage that an organism reads (decodes, decrypts, transcribes) as a template for ordering the structures of biomolecules that an organism synthesizes to impart regulated function to the organism. Chemically, a gene is a heteropolymer comprised of subunits (“nucleotides”) arranged in a specific sequence. In cells, such heteropolymers are deoxynucleic acids (“DNA”) or ribonucleic acids (“RNA”). DNA forms long strands. Characteristically, these strands occur in pairs. The first member of a pair is not identical in nucleotide sequence to the second strand, but complementary. The tendency of a first strand to bind in this way to a complementary second strand (the two strands are said to “anneal” or “hybridize”), together with the tendency of individual nucleotides to line up against a single strand in a complementarily ordered manner accounts for the replication of DNA. Experimentally, nucleotide sequences selected for their complementarity can be made to anneal to a strand of DNA containing one or more genes. A single such sequence can be employed to identify the presence of a particular gene by attaching itself to the gene. This so-called “probe” sequence is adapted to carry with it a “marker” that the investigator can readily detect as evidence that the probe struck a target.

Alternatively, such sequences can be delivered in pairs selected to hybridize with two specific sequences that bracket a gene sequence. A complementary strand of DNA then forms between the “primer pair.” In one well-known method, the “polymerase chain reaction” or “PCR,” the formation of complementary strands can be made to occur repeatedly in an exponential amplification. A specific nucleotide sequence so amplified is referred to herein as the “amplicon” of that sequence. “Quantitative PCR” or “qPCR” herein, refers to a version of the method that allows the artisan not only to detect the presence of a specific nucleic acid sequence but also to quantify how many copies of the sequence are present in a sample, at least relative to a control. As used herein, “qRTPCR” may refer to “quantitative real-time PCR,” used interchangeably with “qPCR” as a technique for quantifying the amount of a specific DNA sequence in a sample. However, if the context so admits, the same abbreviation may refer to “quantitative reverse transcriptase PCR,” a method for determining the amount of messenger RNA present in a sample. Since the presence of a particular messenger RNA in a cell indicates that a specific gene is currently active (being expressed) in the cell, this quantitative technique finds use, for example, in gauging the level of expression of a gene. Collectively, the genes of an organism constitute its genome.

Statistical methods are typically used for determining the predictive models, as well as determine the quality of these prediction models, including plant phenotype predictors, and such methods are well known in the art.

The plant phenotype predictors presented here have been generated with two classes of statistical models: regression models and classification models. The regression models aim to predict the exact continuous value of the phenotype of interest (e.g., exact leaf size), while the classification models output a discretized value for the phenotype of interest (e.g., small, medium or large leaf size).

The evaluation of these prediction models is done using the measures “correlation” and accuracy, respectively, for regression and classification models.

The term “correlation,” as used herein, belongs to the field of statistics. The general meaning of the term “correlation,” is well known in the art. In general, “correlation” is known to indicate the strength and direction of a relationship, in most cases a more or less linear relationship, between two (random) variables. Thus, applied to the present disclosure, the two (random) variables, to which the term “correlation” in the generally known sense refers, are, firstly, the output of a plant phenotype predictor and, secondly, the (potential for) expression of a (future) phenotype. The term “accuracy” refers to the predictive quality of a classification model, obtained by comparing the discretized output labels of the prediction model to the true output labels, thereby counting the number of correctly predicted output labels.

Accordingly, the results of a method for determining predictive quality, as disclosed herein, provides the information if and how differences in (the potential for) expression of a (future) phenotype of (a) plant(s) are reflected by the differences in the plant phenotype predictor based on the plant(s). A non-limiting example for “determining the predictive quality of the plant phenotype predictor,” according to the disclosure, is provided herein and is described in the appended examples. From these examples, the plant phenotype to be observed exemplarily was leaf organ size. The term “evaluation analysis,” as used herein, refers to any (statistical) analysis approach suitable to obtain the “predictive quality,” as defined herein. Accordingly, it is envisaged that the “evaluation analysis” to be performed in the context of this disclosure is suitable to find out if and how the plant phenotype predictor and the (potential for) expression of a (future) phenotype correlate. Since a plant phenotype predictor is based on multiple gene expression values, as described herein before, an “evaluation analysis” “suitable” to be employed herein, is capable to determine a “correspondence” between multiple variables (like multiple gene expression values) on the one hand and a single variable (e.g., like the (potential for) expression a certain (future) phenotype of a plant) on the other hand. Such “evaluation analysis” comprises correspondingly applicable statistical methods. Based on his common general knowledge and the disclosure provided herein, the skilled person is readily in a position to find out evaluation analysis methods, and hence, correspondingly applicable statistical methods that are suitable to be employed in the context of the present disclosure. Examples for such evaluation analysis methods are described herein and are given in the appended examples.

The predictive models “suitable” to be employed herein, particularly, are models that result in a mathematical function between a gene expression signature and the expression of a phenotype.

These models consist of both regression models and classification models, and are able to perform a multivariate analysis. For example, such regression methods include multivariate linear regression analysis, canonical correlation analysis (CCA), an ordinary least square (OLS) regression analysis, a partial least squares (PLS) regression analysis, principal component regression (PCR) analysis, ridge regression analysis, Support Vector regression analysis, decision tree based model regression method, Random Forest regression model, a least absolute shrinkage and selection (LASSO) regression model, a neural network based regression model, or a least angle regression (LAR) analysis.

In the case of classification models, examples include linear and nonlinear support vector machines (SVMs), decision trees, Random Forests, Neural Networks or Bayesian classifiers.

In this context, the skilled person is readily in the position to find out suitable methods to be applied correspondingly. As used herein, the term “evaluating” a plant phenotype predictor based on the “correlation” or accuracy determined by the corresponding methods of the present disclosure means that a given determined plant phenotype predictor, for which the (potential for) expression of a desired (future) phenotype is to be determined, is related to the results/outcome of these methods. The skilled person is readily in the position to put the step of “evaluating” into practice based on his common general knowledge and the teaching provided herein. The result of the evaluation analysis to be employed in the context of the present disclosure can be described as the best possible model, resulting in the highest correspondence between model predictions and the particular (future) phenotype to be observed. For the evaluation step of a specific plant phenotype predictor to be employed herein, any suitable analysis method can be used. The skilled person is readily in the position to find out such suitable analyses methods by his common general knowledge and the teaching provided herein. As a non-limiting example, such an analysis approach can be employed as it is exemplified in the appended examples.

As mentioned above, based on his common general knowledge and the teaching provided herein, a skilled person is readily in the position to find out “evaluation analyses” as well as “evaluating” and “deducing” approaches suitable to be employed in the context of the present disclosure. As mentioned, such analyses and approaches involve suitable statistical analyses of the data obtained in the context of the methods of the present disclosure. This refers to any mathematical analysis method that is suited to further process the data obtained. For example, these data represent the amounts of the analyzed gene expression values present in a plant phenotype predictor present in a tissue, either in absolute terms (e.g., fluorescence values) or in relative terms (i.e., normalized to a certain reference quantity), the results of the analyses of the correspondence between the plant phenotype predictor and the (potential for) expression of a (future) phenotype as provided and described herein, and/or the determined (potential for the) expression of a (future) phenotype to be observed. Mathematical methods and computer programs to be applied in context of the statistical analyses to be employed in the context of this disclosure can be found out by the skilled practitioner. Examples include SAS, SPSS and R. In yet another embodiment, the statistical analyses to be employed in the context of the methods of the disclosure takes into account higher order gene dependencies, which may lead to improved perfoimance of the prediction models.

In yet another embodiment, the disclosure provides a method for selecting a suitable plant genotype comprising a phenotype of interest for the introduction of a trait expressing a phenotype related to the phenotype of interest, the method comprising the following steps: i) providing a genotype collection of immature plants displaying a variation of a phenotype of interest related to the phenotype expressed by the trait wherein the phenotype is only visible when the plants are mature, ii) isolating a tissue from each immature plant in the genotype collection wherein the tissue is determinative for the phenotype, iii) carrying out a transcriptional profile on each of the tissues, iv) evaluating the correspondence between a plant phenotype predictor present in the transcriptional profile and the plant phenotype of interest with a statistical model, the correspondence being previously measured by a) providing a reference genotype collection of immature plants displaying a variation of the phenotype of interest, b) isolating a tissue from each immature plant in the genotype collection wherein the tissue is determinative for the phenotype, c) carrying out a transcriptional profile on each of the tissues and d) determining a plant phenotype predictor associated with the phenotype of interest, based on the evaluation in step iv) selecting a suitable plant genotype for the introduction of a trait encoding a specific phenotype.

Plant phenotype predictors have been described herein before.

In a particular embodiment, the trait is introduced via breeding. In yet another particular embodiment, the trait is introduced via transformation.

In another embodiment, the trait is a recombinant trait.

In yet another embodiment, the trait is a natural trait. A “natural trait” is equivalent with the term “native trait.”

In another particular embodiment, the “suitable plant genotype” is a suitable germplasm entry derived from a plant germplasm collection.

In yet another specific embodiment, the method for the selection of a suitable plant genotype further comprises the making of a plant breeding decision based on the association of at least one plant genotype with the performance of at least one transgenic trait expressing a phenotype related to the phenotype of interest. In yet another particular embodiment, the selected plant genotype, in particular a selected geiniplasm entry, is used in making a breeding cross. In yet another specific embodiment, the selected gennplasm entry is used as a donor to introgress a genomic region into at least one recipient germplasm entry.

The wording “a trait expressing a phenotype related to the phenotype of interest” means that the trait (either natural or recombinant) when introduced in a plant (via crossing or transformation) leads to the expression of the trait in the plant and the expression has an effect on the plant phenotype of interest. The latter means that when the trait is expressed in the plant that the phenotypic outcome of the expression of the trait in the plant influences the phenotype of interest in the plant. “Influences” can mean enhances, stimulates, lowers, diminishes, reduces or synergizes. In a particular embodiment, a recombinant trait can comprise a (or more than one) member of the constituents (i.e., a gene) of the identified plant phenotype predictor, which was found associated with a plant phenotype. Such a gene can, for example, form part of a plant recombinant vector and introduced into a plant (e.g., by transformation). In another particular embodiment, a recombinant trait does not comprise a member of the constituents of the identified plant phenotype predictor.

In yet another embodiment, the disclosure provides a method for obtaining a biological or chemical compound, which is capable of generating a plant with a phenotype of interest comprising i) providing a collection of immature plants, ii) subjecting the population of plants with a biological or chemical compound, iii) obtaining a nucleic acid sample from a tissue from each of the plants wherein the tissue is determinative for the phenotype, iv) carrying out a transcriptional profile on each of the tissues, v) evaluating the correspondence between a plant phenotype predictor present in the transcriptional profile and the plant phenotype of interest with a statistical method, the correspondence being previously measured by a) providing a reference collection of immature plants displaying an expected variation of the phenotype of interest, b) isolating a tissue from each of the plants present in the reference collection, c) carrying out a transcriptional profile on each of the tissues, and d) determining a plant phenotype predictor present in the transcriptional profile, which is associated with the phenotype, and vi) based on the evaluation in step v) selecting a plant comprising a phenotype of interest.

In step ii) any biological or chemical compound may be contacted with the plants. It is also envisaged that a plurality of different compounds can be contacted in parallel with plants. Preferably, each test compound is brought into physical contact with one or more individual plants. Contact can also be attained by various means, such as spraying, spotting, brushing, applying solutions or solids to the soil, to the gaseous phase around the plants or plant parts, dipping, etc. The test compounds may be solid, liquid, semi-solid or gaseous. The test compounds can be artificially synthesized compounds or natural compounds, such as proteins, protein fragments, volatile organic compounds, plant or animal or microorganism extracts, metabolites, sugars, fats or oils, microorganisms such as viruses, bacteria, fungi, etc. In a preferred embodiment, the biological compound comprises or consists of one or more microorganisms, or one or more plant extracts or volatiles (e.g., plant headspace compositions). The microorganisms are preferably selected from the group consisting of: bacteria, fungi, mycorrhizae, nematodes and/or viruses. It is especially preferred and evident that the microorganisms are non-pathogenic to plants, or at least to the plant species used in the method. Especially preferred are bacteria which are non-pathogenic root colonizing bacteria and/or fungi, such as Mycorrhizae. Mixtures of two, three or more compounds may also be applied to start with, and a mixture which shows an effect on priming can then be separated into components which are retested in the method. Using mixtures, also synergistically acting compounds can be identified, i.e., compounds, which provide a stronger priming effect together than the sum of their individual priming effect. Preferably, compositions are liquid or solid (e.g., powders) and can be applied to the soil, seeds or seedlings or to the aerial parts of the plant.

In yet another embodiment, the disclosure provides a plant phenotype predictor indicative for a plant phenotype of interest. In another embodiment, the plant phenotype predictor is used for the selection of a plant comprising a phenotype of interest, according to the methods described herein.

In yet another embodiment, the plant phenotype predictor is used in the method for obtaining a biological or chemical compound, which is capable of generating a plant with a phenotype of interest.

In another aspect, the disclosure is embodied in a kit useful for detecting a plant phenotype predictor correlated with a phenotype of interest. To effectively detect a plant phenotype predictor in a tissue derived from an immature plant, which is characteristic for a plant with a phenotype of interest, the expression level of the genes present in the plant phenotype predictor needs to be measured. A kit to carry out a PCR analysis, preferably a multiplex PCR analysis such as a multiplex RT-PCR analysis comprises primers, buffers, polynucleotides and a thermostable DNA polymerase. Another kit is a microarray comprising the nucleotide sequences derived from the genes, which are the constituents of the plant phenotype predictor.

In a particular embodiment, based on the identified plant phenotype predictor, it is possible to determine an alternative plant phenotype predictor. In a particular embodiment, a plant phenotype predictor profile can also be detected by the use of specific antibodies directed against the protein products encoded by the genes present in plant phenotype predictor. Such an application can also be embodied in a kit such as, for example, a protein array.

In yet another embodiment, the disclosure provides a set of plant phenotype predictors for leaf biomass production of which the constituents of the plant phenotype predictors are presented in Table 5. As an example for a particular plant phenotype predictor for leaf growth (derived from Table 5), genes 1 is IAA16, gene 2 is GNC and gene 3 AtGRF5.

The methods and means described herein, are believed to be suitable for all plant cells and plants, gymnosperms and angiosperms, both dicotyledonous and monocotyledonous plant cells and plants including, but not limited to, Arabidopsis, alfalfa, barley, bean, corn, cotton, flax, oat, pea, rape, rice, rye, safflower, sorghum, soybean, sunflower, tobacco and other Nicotiana species, including Nicotiana benthamiana, wheat, asparagus, beet, broccoli, cabbage, carrot, cauliflower, celery, cucumber, eggplant, lettuce, onion, oilseed rape, pepper, potato, pumpkin, radish, spinach, squash, tomato, zucchini, almond, apple, apricot, banana, blackberry, blueberry, cacao, cherry, coconut, cranberry, date, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut and watermelon Brassica vegetables, sugarcane, vegetables (including chicory, lettuce, tomato), Lemnaceae (including species from the genera Lemna, Wolffiella, Spirodela, Landoltia, Wolffia) and sugar beet.

The following non-limiting examples describe methods and means according to the disclosure. Unless stated otherwise in the examples, all techniques are carried out according to protocols standard in the art. The following examples are included to illustrate embodiments of the disclosure. Those of skill in the art should, in light of the present disclosure, appreciate that many changes can be made in the specific embodiments, which are disclosed and still obtain a like or similar result without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure, as defined by the appended claims.

### EXAMPLES

### Gene Selection Based on Expression Profiling of Early Leaf Development

To assess the changes in the transcriptome during early leaf development, we profiled gene expression in leaf tissues using AGRONOMICS1 tiling arrays (Andriankaja et al., 2012). The third true leaf of Arabidopsis was harvested daily from 8 to 13 days after stratification. At day 8 and day 9, the third leaf was entirely composed of proliferating cells, whereas beginning at day 10, the leaf began to transition with the cells in the tip of the leaf starting to expand, while the cells in the base continued to proliferate. This gradient of cell proliferation and expansion persisted through day 11 and 12, and then, at day 13 the majority of cells in the base of the leaf also began to expand. The transcriptome profiling allowed for the identification of over 9664 genes that were differentially regulated between at least two consecutive time points. 458 genes encode transcription factors (TF) based on AGRIS (on the World Wide web at arabidopsis.med.ohio-state.edu/) and Gene Ontology, while 286 of these transcription factor-encoding genes show a difference in expression of at least two fold between any two time points.

These 286 TFs were further reduced to a set of so-called growth predictors based on co-expression analysis. First, these genes were divided in subsets according to their specific temporal expression pattern in the early leaf development tiling array data. For subsets with a large number of TFs, additional microarray expression data on developing leaves was used to identify clusters of tightly co-expressed genes. These microarray data comprise experiments assessing early leaf development in standard and mild drought stress conditions. Finally, representative genes for each cluster were chosen based on prior knowledge, resulting in a final list of 98 growth predictors. For the nCounter experiment, 10 housekeeping genes were added to yield a total list of 108 genes (see Table 2).

2. Correlation Between Initial Leaf Size and Final Leaf Size

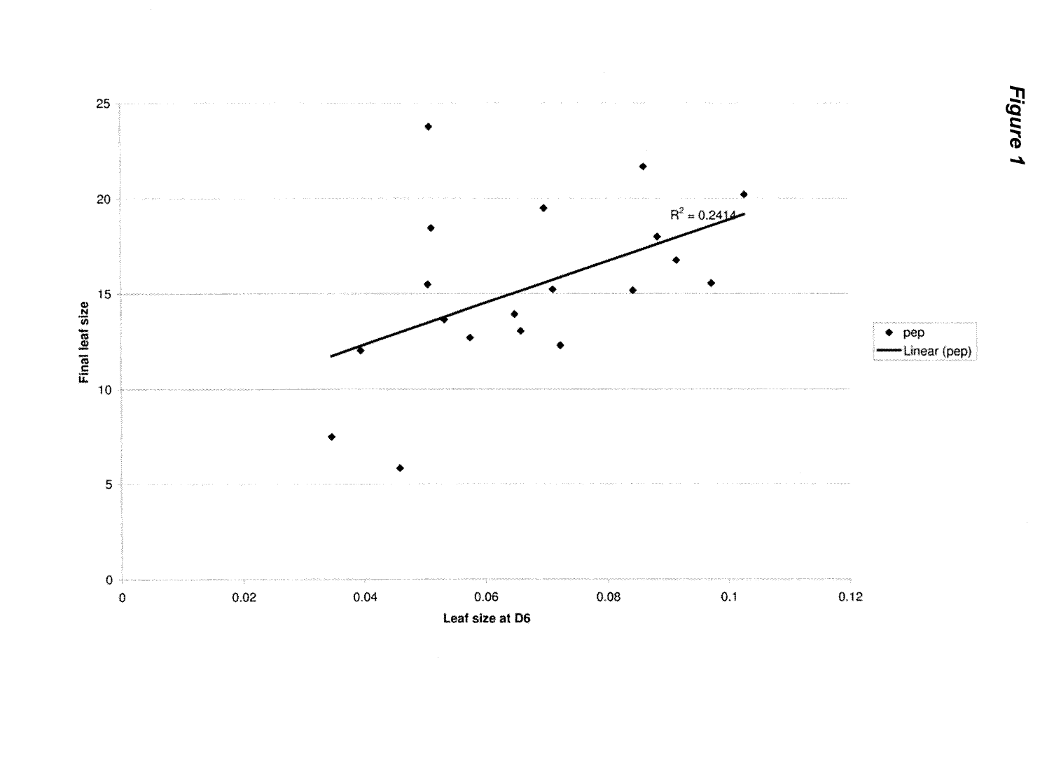

We have measured the size of leaf 1 and 2 at harvest (D6) and at maturity (D21). FIG. 1 presents the correlation between the initial leaf size (when leaves are harvested for expression profiling) and final leaf size (that we want to predict based on the expression profile at D6). Initial and final leaf size are only linked in some cases. Therefore, the initial leaf size cannot be used to predict the final leaf size. We will show below that, instead, the expression profile determined from leaves harvested at D6 is predictive for final leaf size.

3. Prediction of Leaf Growth Phenotypes Through Classification

Phenotypic classes are determined based on final leaf size of the plants with altered leaf size due to the overexpression or knock-out of one or more genes.

63 samples were classified in three classes, namely “SMALL (S),” “NORMAL (N),” “LARGE (L),” based on the final leaf size (size of leaf 1 and 2 at maturity). Class S contains AN3_D6, APC10_D6, Col09_D6, Col_DA1_D6, Col_GOLS2 D6, GA3OX1_D6, GOLS2_D6, SCR_D6, class N contains bHLH101_D6, BRI1_D6, Col_GA3ox_D6, JAW_D6, SAUR19_D6, and class L contains Col_ami_PPD_D6, DA 1-1_D6_run1, DA1-1_D6_run2, DA1-1_EOD_D6_run1, DA1-1_EOD_D6_run2, EOD_D6, GRA_D6, GRF5_D6 (three biological replicates).

Machine learning approaches such as state-of-the-art support vector machines (SVM) are used for the classification of samples based on transcript activities concordant with the phenotypic parameters.

4. Evaluation of Classification

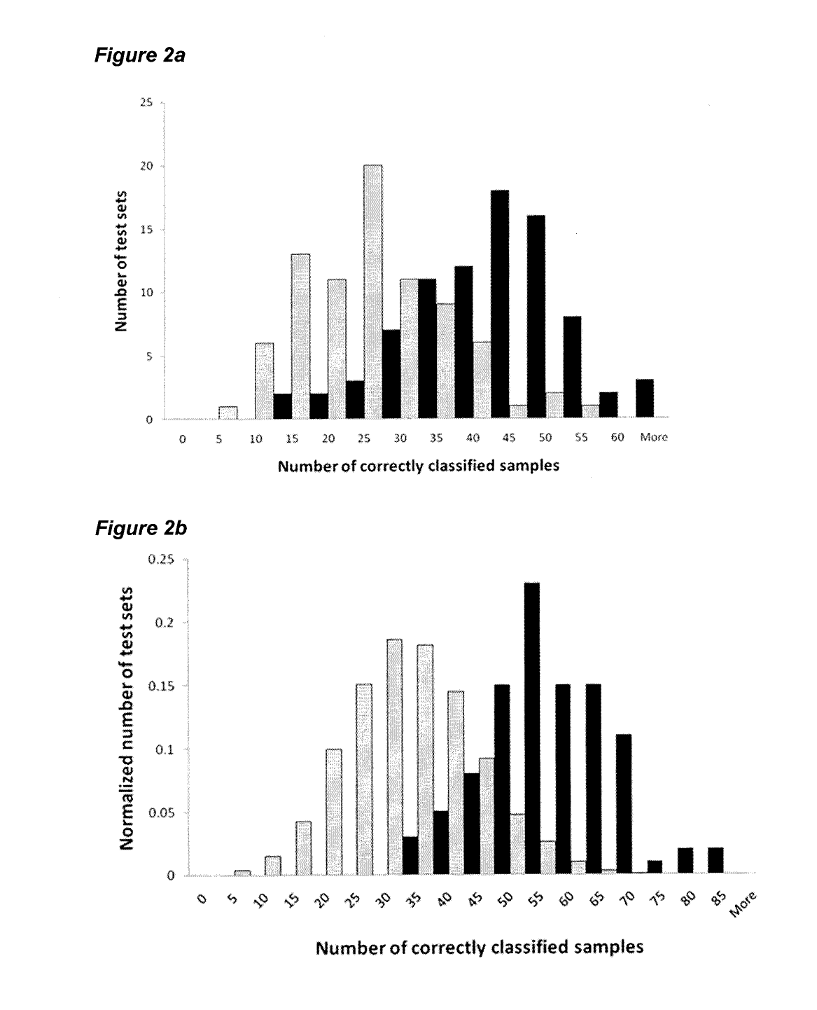

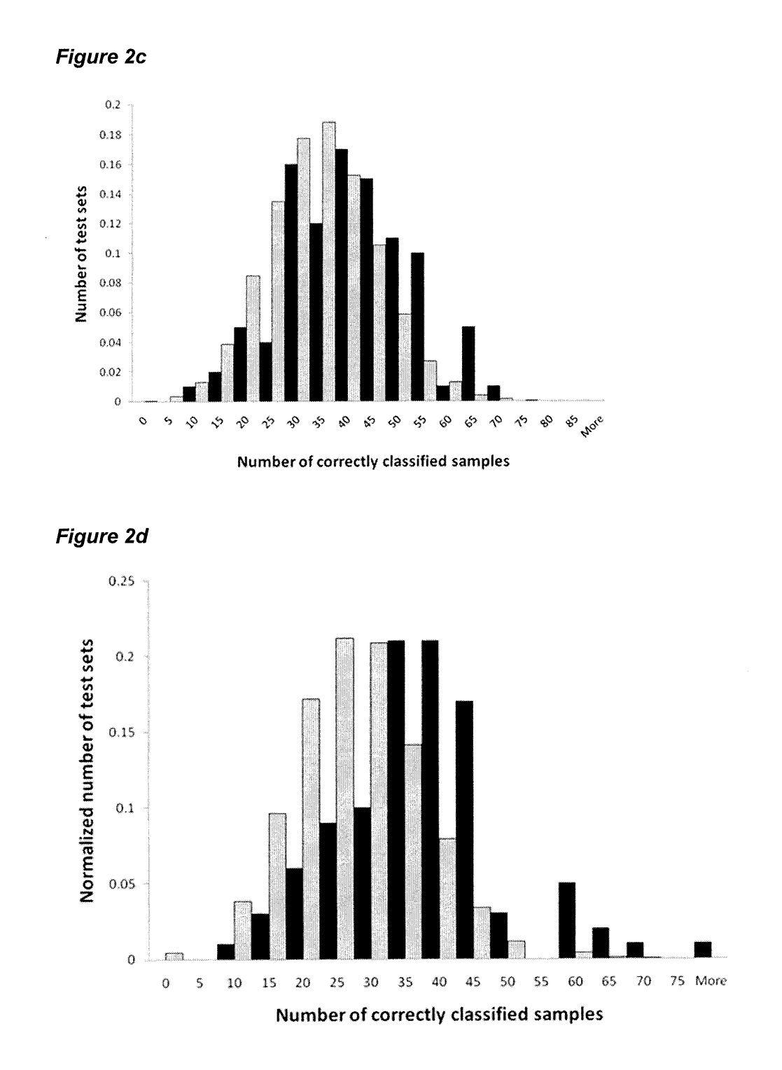

Separate training and test sets were generated to rigorously evaluate the classification through support vector machines (WEKA SMO function). 66% of the samples were used as training data, while 33% of the samples were used as test data. Each time, all three replicates of a sample were assigned to either the training or the test set and at least 3 (×3) samples of each classes were used as training data. The construction of these sets was repeated 100 times to estimate the variability in classification error depending on the specific samples in the training and/or test dataset. In addition, class labels were permuted to generate randomized training and test datasets. Comparing the percentage of correctly classified samples in the real and randomized datasets shows a significant difference between their score distributions (mean real=41%, mean random=24%, p-value <2.2e-16) (see FIG. 2a).

In addition to final leaf size, the phenotypic parameters leaf size at harvest and final rosette area, or any other phenotype, can be used as a target for classification. Prediction of the size of the leaf at the time of harvesting for RNA extraction perfoinis considerable better (see FIG. 2b, mean real=54.6%, mean random=31.4%, p-value <2.2e-16). Prediction of the final rosette size is relatively difficult), which is most probably due to the difficulty in automatically extracting this phenotypic parameter from the images. However, a significant difference between the real and random datasets is observed (see FIG. 2c, mean real=38.6%, mean random=32.4%, p-value=9.186e-07.

Alternative to a classification based on leaf size, the different transgenic lines can be classified based on the cellular mechanism by which differences in leaf size are obtained. Growth is controlled through a combination of cell division and cell expansion. With the current knowledge, leaf growth can be best described as the succession of five overlapping and interconnected phases: an initiation phase, a general cell division phase, a transition phase, a cell expansion phase, and a meristemoid division phase. The analysis of transgenic lines with altered leaf size suggests that at least four of the five mechanisms contribute to the final leaf size (Gonzalez et al., 2012).

Based on these cellular mechanisms, the transgenic lines in this study are classified as follows: class A contains the different control lines (Col09_D6, Col_DA1_D6, Col_GOLS2_D6, Col_ami_PPD_D6, Col_GA3ox_D6), class B contains transgenic lines that show faster leaf growth (APC10_D6, DA1-1_D6_run1, DA1-1_D6_run2, DA1-1_EOD_D6_run1, DA1-1_EOD_D6_run2), class C contains transgenic lines having a longer time of cell proliferation (GRF5_D6, EOD_D6, GRA_D6, JAW_D6) and class D contains transgenic lines that have smaller leaves due to a lower number of cells (AN3_D6, GA3OX1_D6, SCR_D6).

The evaluation of the classification was performed, as described for the prediction of final leaf size. A summary of the results of classification based on mechanism can be found in FIG. 2d. A significant difference between the score distributions of real and random data (mean real=35.8%, mean random=22.8%, p-value <2.2e-16) is observed.

5. Prediction of Leaf Growth Phenotypes Through Regression

Regression methods such as linear regression are used to link expression and phenotype profiles without prior classification of the samples based on the measured phenotype. For each analysis, leave-one-out cross-validation was done, using the Pearson correlation coefficient between the observed and predicted phenotype profile as a performance measure.

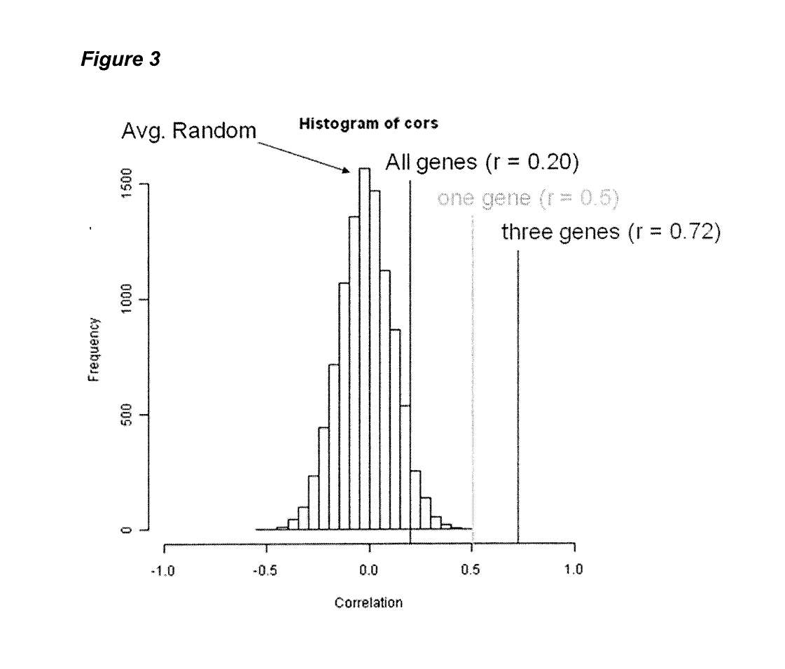

In a first step, single gene regression models were constructed. For each model, a p-value was calculated using a label permutation test to assess the significance of the resulting predictions. Table 3 shows the top ranked single gene models, as well as their correlation and p-values. Subsequently, all pairs of genes were explored, trying to improve the correlation by looking at combinatorial effects. Table 4 shows the top ranked pairwise gene models, including their correlation and p-values. Finally, we explored models consisting of triplets of genes by looking at the top ranked genes in the list of pairwise gene models. From the top 15 performing genes, all triplet combinations were made, which are shown in Table 5. FIG. 3 summarizes the regression analysis. The figure shows the distribution of correlations for random regression models, the regression model using all genes (blue line), using the best single gene model (green line), and the best triplet model (red line). Combinations of more than 3 genes did not improve the predictions. In accordance, using all profiled genes or genes identified through feature selection results in poorer predictions of leaf size.

6. Pinpointing Key Leaf Growth Regulators

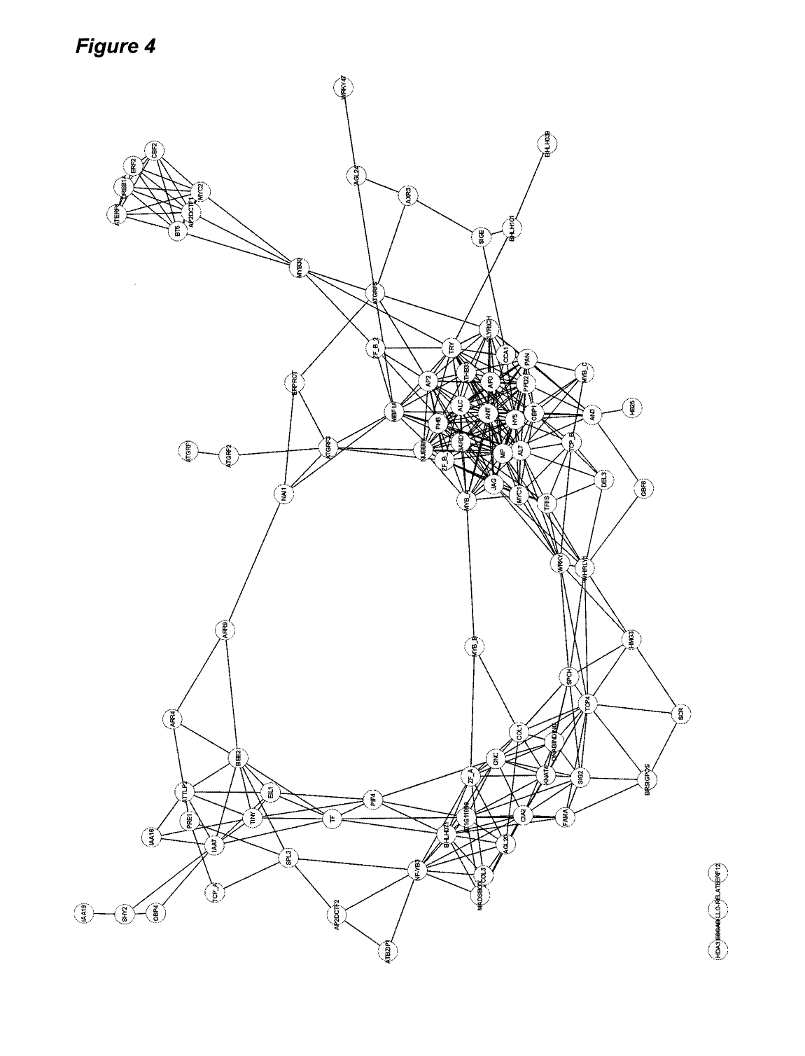

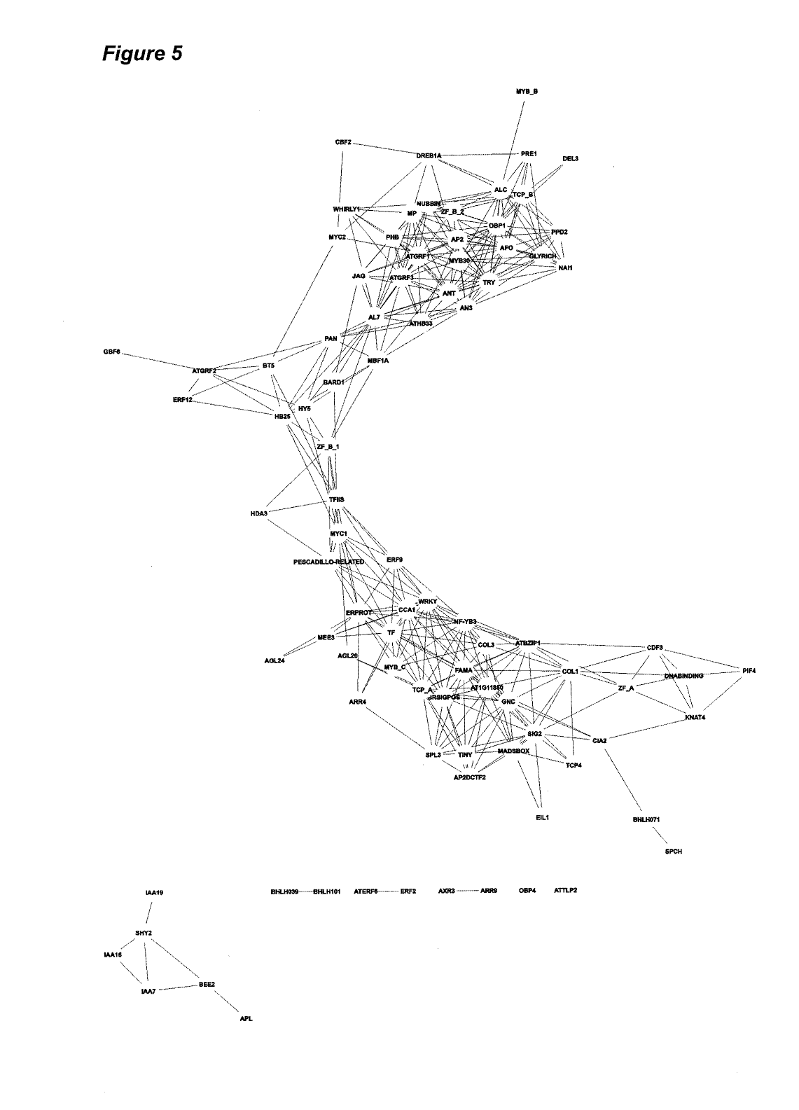

Based on the available expression data, the similarity in expression between the different putative growth regulators is assessed. By studying co-expression networks, the validity of a gene or any of its co-expressed genes in a prediction model is investigated. Moreover, co-expression network analysis allows to distinguish different clusters of genes with similar expression behavior. Finally, co-expression is calculated based on different subsets of the expression data, thereby identifying differential co-expression networks. Subsetting of the expression data is done based on the final leaf size sample classes (see FIGS. 4 and 5). Subsequently, we can test whether two genes and/or a cluster of genes co-express in all subsets of the expression data. Hereby, we can pinpoint relevant changes in genes related to differences between sample classes.

73 pairs of growth predictors are co-expressed (PCC >0.65) in all subsets of the expression data (small, normal and large). For instance, BHLH039 and BHLH101, CBF2 and DREBIA, or ANT and AFO are co-expressed in all size classes of plants, while, for instance, MYC2 and ATERF6 are co-expressed in small and normal sized plants, but not in large plants, and ANT and TINY show negatively correlated expression patterns in small and large plants and are not correlated in normal sized plants.

TABLE 1
Arabidopsis transgenic lines and conditions.

| | | | treat- | | | |
|---|---|---|---|---|---|---|
| line | AGI | condition | ment | leaf | age | modification |
| Col-0 | — | in vitro-1 | — | 1 + 2 | D6 | — |
| Col_GOL | — | in vitro-2 | — | 1 + 2 | D6 | — |
| S2 | | | | | | |
| Col_DA | — | in vitro-2 | — | 1 + 2 | D6 | — |
| Col_GA3 | — | in vitro-2 | — | 1 + 2 | D6 | — |
| Col_PPD | — | In vitro-2 | — | 1 + 2 | D6 | — |
| Col_other | — | In vitro-2 | — | 1 + 2 | D6 | — |
| da1-1 | AT1G19270 | in vitro-12 | — | 1 + 2 | D6 | LOF |
| da1-1/eod1 | AT1G19270/ | in vitro-12 | — | 1 + 2 | D6 | LOF |
| | AT3G63530 | | | | | |
| eod1 | AT3G63530 | in vitro-2 | — | 1 + 2 | D6 | LOF |
| GRF5 | AT3G13960 | in vitro-2 | | 1 + 2 | D6 | GOF |
| BRI1 | AT4G39400 | in vitro-2 | | 1 + 2 | D6 | GOF |
| AN3 | AT5G28640 | in vitro-2 | — | 1 + 2 | D6 | LOF |
| APC10 | AT2G18290 | in vitro-2 | — | 1 + 2 | D6 | GOF |
| bhlh101 | AT5G04150 | in vitro-2 | — | 1 + 2 | D6 | LOF |
| ga3ox1 | AT1G15550 | in vitro-2 | — | 1 + 2 | D6 | LOF |
| GOLS2 | AT1G56600 | in vitro-2 | — | 1 + 2 | D6 | OE |
| gra | | in vitro-2 | — | 1 + 2 | D6 | segm dupl |
| JAW | AT4G23713 | in vitro-2 | — | 1 + 2 | D6 | OE |
| SAUR19- | | in vitro-2 | — | 1 + 2 | D6 | OE |
| GFP | | | | | | |
| SCR | AT3G54220 | in vitro-2 | — | 1 + 2 | D6 | LOF |

LOF: loss of function,
GOF: gain of function,
OE: overexpression (35S)

TABLE 2
List of phenotype predictors

AT1G04020
AT1G04240
AT1G04250
AT1G08540
AT1G09250
AT1G10470
AT1G11850
AT1G13400
AT1G14410
AT1G14510
AT1G19850
AT1G22510
AT1G22590
AT1G28360
AT1G30490
AT1G32640
AT1G34310
AT1G63100
AT1G68480
AT1G68640
AT1G75240
AT1G79430
AT2G18280
AT2G21650
AT2G22770
AT2G22840
AT2G24790
AT2G27050
AT2G31730
AT2G33810
AT2G36080
AT2G36400
AT2G38560
AT2G42680
AT2G43010
AT2G44940
AT2G45190
AT2G45660
AT2G46830
AT3G01330
AT3G04730
AT3G09600
AT3G13040
AT3G13960
AT3G15030
AT3G15540
AT3G16870
AT3G23050
AT3G24140
AT3G28910
AT3G44750
AT3G47500
AT3G50410
AT3G50750
AT3G56980
AT3G57040
AT4G00480
AT4G01720
AT4G14540
AT4G14720
AT4G17490
AT4G23800
AT4G24540
AT4G25470
AT4G25480
AT4G29030
AT4G31805
AT4G34590
AT4G36540
AT4G36920
AT4G37610
AT4G37740
AT4G37750
AT5G04150
AT5G08330
AT5G11060
AT5G11260
AT5G14520
AT5G15850
AT5G17300
AT5G24120
AT5G28640
AT5G39860
AT5G44210
AT5G46690
AT5G47220
AT5G47610
AT5G49450
AT5G51190
AT5G51910
AT5G53200
AT5G53210
AT5G56860
AT5G57180
AT5G60850
AT5G61590
AT5G65410
AT5G67110

TABLE 3
Single gene regression models

| Gene | PCC | p-value |
|---|---|---|
| TINY | 0.505211698764495 | 0 |
| IAA16 | 0.486731885403237 | 0 |
| AN3 | 0.398464581784203 | 1e−04 |
| HB25 | 0.389319818331105 | 6e−04 |
| TF | 0.378171745533332 | 6e−04 |
| ANT | 0.364025362881023 | 9e−04 |
| OBP1 | 0.362864017510679 | 0.0013 |
| AT1G11850 | 0.346080531443792 | 0.0015 |
| GNC | 0.324756090674234 | 0.0016 |
| IAA7 | 0.308588579442458 | 0.0032 |
| origpep | 0.301836003162092 | 0.0045 |
| EIL1 | 0.257409772502492 | 0.0074 |
| MP | 0.246396166152757 | 0.0096 |
| MADSbox | 0.239025253739644 | 0.0112 |
| WHIRLY1 | 0.178770319120969 | 0.0321 |
| PAN | 0.13950643169144 | 0.0491 |

TABLE 4
Regression models of two genes

| Gene 1 | Gene2 | Correlation | p-value |
|---|---|---|---|
| IAA16 | GNC | 0.66864165065833 | 0 |
| OBP1 | NAI1 | 0.655293607738098 | 0 |
| WHIRLY1 | GNC | 0.634757870235222 | 0 |
| IAA16 | AtGRF5 | 0.628015438573879 | 0 |
| GNC | OBP4 | 0.622161356587898 | 0 |
| AT1G11850 | IAA16 | 0.616342904838885 | 0 |
| AT1G11850 | NAI1 | 0.615195667463601 | 0 |
| NUBBIN | OBP1 | 0.611556444232159 | 0 |
| ANT | NAI1 | 0.605574895959673 | 0 |
| AXR3 | IAA16 | 0.605414372810791 | 0 |
| HMG3 | GNC | 0.59081489607892 | 0 |
| TRY | NAI1 | 0.590638869032686 | 0 |
| IAA16 | CIA2 | 0.587735616227911 | 0 |
| IAA16 | SPCH | 0.585331922058697 | 0 |
| IAA16 | FAMA | 0.58523241970164 | 0 |

TABLE 5
Regression models of three genes

| Gene1 | Gene2 | Gene3 | Correlation | p-value |
|---|---|---|---|---|
| IAA16 | GNC | AtGRF5 | 0.72468475818384 | 0 |
| GNC | OBP1 | NAI1 | 0.718149385957251 | 0 |
| IAA16 | OBP1 | NUBBIN | 0.716380844442301 | 0 |
| OBP1 | NAI1 | NUBBIN | 0.712635651845443 | 0 |
| IAA16 | GNC | OBP4 | 0.703465074952496 | 0 |
| OBP1 | NAI1 | AtGRF5 | 0.69503440047168 | 0 |
| GNC | WHIRLY1 | NUBBIN | 0.692484200044535 | 0 |
| GNC | NAI1 | OBP4 | 0.692148706259356 | 0 |
| ANT | WHIRLY1 | NUBBIN | 0.691267634842454 | 0 |
| IAA16 | GNC | NAI1 | 0.690202644054748 | 0 |
| GNC | NAI1 | WHIRLY1 | 0.68751030612779 | 0 |
| GNC | OBP4 | HMG3 | 0.684767664068834 | 0 |
| IAA16 | AtGRF5 | AXR3 | 0.683013592051983 | 0 |
| IAA16 | GNC | WHIRLY1 | 0.679391341575104 | 0 |
| WHIRLY1 | AT1G11850 | NUBBIN | 0.677191270948943 | 0 |
| IAA16 | GNC | HMG3 | 0.675982245557348 | 0 |

### Materials and Methods

Leaf Growth Mutants

Samples contain transgenic plants in which a particular gene was overexpressed or mutated. All mutants are grown in vitro and have a Columbia background.

The transgenic lines can be divided in two categories:

The category of smaller plants corresponds to transgenics in which the expression of the following genes was modified: AN3, bHLH101, GOLS2, GA30×1, SCR.

The an 3 loss of function mutants produce leaves that are narrower than those of wild type and contain less but larger cells (Horiguchi et al., 2005). Downregulation of bHLH101 also leads to production of smaller leaves (unpublished data), although previously this transgenic line was described to have no leaf size difference compared to wild type plants (Wang et al., 2007). Plants overexpressing GOLS2 produce smaller leaves (unpublished data). Finally, in the scarecrow (SCR) mutants, leaves are smaller due to a reduced cell division rate and early exit of the proliferation phase (Dhondt et al., 2010). The ga3ox1-3 loss of function mutant has lower GA levels and consequently impaired leaf growth (Mitchum et al., 2006).

The category of larger plants corresponds to transgenics in which the expression of the following genes was modified: APC10, BRI1, DA1, EOD, DA-EOD, GRA, GRF5, JAW, SAUR19.

Plants overexpressing APC10 produce larger leaves containing more cells (unpublished data). The overexpression of BRI1 under the control of its own promoter leads to the formation of longer leaves containing more cells (Gonzalez et al., 2010). In the mutant dal-1, leaves are larger and contain more cells (Li et al., 2008). The downregulation of EOD/BB also leads to the production of larger organs (Li et al., 2008). We also analyzed the expression of these transcription factors in double mutants of dal-1 and eod that show a synergistic effect of leaf size (Li et al., 2008). The grandifolia line that contains a duplication of a part of the chromosome 4 produces larger leaves containing more cells (Horiguchi et al., 2009). Overexpression of GRF5 leads to the formation of larger leaves containing more cells (Horiguchi et al., 2005; Gonzalez et al., 2010). Plants overexpressing the miRNA JAW produce larger leaves due to an increase in cell proliferation at the edge of the leaf (Palatnik et al., 2003). Finally, plants overexpressing the SAUR19 genes fused to a GFP tag produce larger leaves containing larger cells (unpublished data, patent).

2. Growth Conditions

Arabidopsis plants were grown for 6 days after stratification (DAS) with a 16 hour day and 8 hour night regime. These were then harvested when leaf 1 and 2 are approximately 0.25-0.35 mm in length from base to tip.

3. Sampling, RNA

The whole plants were harvested by placing them in an excess solution of RNAlater (Ambion) and were then stored at 4° Celsius. Within 10 days, leaf 1 and 2 were removed from these plants by microdissection using a bino microscope and precision microdissection scissors. These microdissections were done on a cool plate to keep the samples from reaching room temperature. Leaf 1 and 2 were collected from at least 200 plants (400 leaves) for each sample and RNA was extracted. The RNA was then checked for quality using the Agilent nano or pico chip (Agilent).

4. Phenotyping

4.1 Leaf 1 and 2 at Time of Harvest

Ten plants from each sample were placed into 100% ethanol for at least 2 hours or until the leaves were cleared. These plants were then transferred to lactic acid and leaf 1 and 2 were removed from each plant using microdissection scissors. The leaves were mounted on slides in lactic acid and then imaged using a bino microscope and differential contrast settings. The images were analyzed for leaf length, width, and area in Image J (on the World Wide web at rsb.info.nih.gov/ij/).

4.2 Leaf 1 and 2 at Maturity (21 Days After Stratification)

A minimum of 6 plants were imaged at 21 days after stratification to determine the mature size of the whole plant and of leaf 1 and 2 only. Leaf 1 and 2 were removed and imaged individually. Leaf areas were analyzed using ImageJ (on the World Wide web at rsb.info.nih.gov/ij/). An average leaf size over at minimum 6 plants was calculated.

5. Generation and Analysis of nCounter Data

A set of 108 genes is profiled using the nCounter technology of NanoString. The nCounter Analysis System (NanoString Technologies, Seattle, Wash., USA) is a fully automated system for digital gene expression analysis (Geiss et al., 2008). The technology enables the multiplexed measurement of individual target RNA molecules. Target mRNAs are detected directly through hybridization to an nCounter Reporter Probe, a molecular barcode. This probe consists of 50 bases, matching the target sequence, to which a series of fluorescent molecules is attached, making up a fluorescent “barcode” that uniquely identifies the target. A second probe of 50 bases, the Capture Probe, matching to the target adjacent to the Reporter Probe, allows immobilization of the mRNA-Probe complex for data collection. In a multiplex reaction up to 800 different target mRNAs can be measured. After hybridization in solution of the probes with the input RNA, excess probes are removed and the probe/target complexes are aligned and immobilized. Using a CCD camera, the presence of the individual barcodes is counted. This allows direct detection of mRNAs using hybridization of probes without reverse transcription or amplification.

The nCounter technology allows to profile such a limited set of genes in a high number of small samples (10 ng of total RNA) at reasonable cost. The technology offers a range of expression of 4 to 5 orders of magnitude, comparable to microarray experiments. Normalization of the nCounter data is done making use of both positive spiked-in controls included by NanoString and control genes (e.g., housekeeping genes) provided by the user. A normalization factor is calculated based upon the most stable housekeeping genes using the GeNorm algorithm (Vandesompele et al., 2002). Rigorous tests have revealed that nCounter is highly sensitive and reproducible (unpublished) (Amit et al., 2009).

### REFERENCES

Amit I, Garber M, Chevrier N, Leite A P, Donner Y, Eisenhaure T, Guttman M, Grenier J K, Li W, Zuk O, Schubert L A, Birditt B, Shay T, Goren A, Zhang X, Smith Z, Deering R, McDonald R C, Cabili M, Bernstein B E, Rinn J L, Meissner A, Root D E, Hacohen N, Regev A (2009) Unbiased reconstruction of a mammalian transcriptional network mediating pathogen responses. Science 326: 257-263
Anastasiou E, Kenz S, Gerstung M, MacLean D, Timmer J, Fleck C, Lenhard M (2007) Control of plant organ size by KLUH/CYP78A5-dependent intercellular signaling. Dev Cell 13: 843-856
Andriankaja M, Dhondt, S., De Bodt, S., Coppens, F., Skirycz, A., Gonzalez, N., Beemster, G. T. S. and Inzé, D. Early leaf development: a not so gradual process. Developmental Cell 22:64-78.
De Veylder L, Beeckman T, Beemster G T, Krols L, Terras F, Landrieu I, van der Schueren E, Maes S, Naudts M, Inze D (2001) Functional analysis of cyclin-dependent kinase inhibitors of Arabidopsis. Plant Cell 13: 1653-1668
Dhondt S, Coppens F, De Winter F, Swamp K, Merks R M, Inze D, Bennett M J, Beemster G T (2010) SHORT-ROOT and SCARECROW regulate leaf growth in Arabidopsis by stimulating S-phase progression of the cell cycle. Plant Physiol 154: 1183-1195
Donnelly P M, Bonetta D, Tsukaya H, Dengler R E, Dengler N G (1999) Cell cycling and cell enlargement in developing leaves of Arabidopsis. Dev Biol 215: 407-419
Eloy N B, de Freitas Lima M, Van Damme D, Vanhaeren H, Gonzalez N, De Milde L, Hemerly A S, Beemster G T, Inze D, Ferrera P C (2011) The apc/c subunit 10 plays an essential role in cell proliferation during leaf development. Plant J 68:351-363.
Gonzalez N, De Bodt S, Sulpice R, Jikumaru Y, Chae E, Dhondt S, Van Daele T, De Milde L, Weigel D, Kamiya Y, Stitt M, Beemster G T, Inze D (2010) Increased leaf size: different means to an end. Plant Physiol 153: 1261-1279
Horiguchi G, Gonzalez N, Beemster G T, Inze D, Tsukaya H (2009) Impact of segmental chromosomal duplications on leaf size in the grandifolia-D mutants of Arabidopsis thaliana. Plant J 60: 122-133
Horiguchi G, Kim G T, Tsukaya H (2005) The transcription factor AtGRF5 and the transcription coactivator AN3 regulate cell proliferation in leaf primordia of Arabidopsis thaliana. Plant J 43: 68-78
Hua J, Meyerowitz E M (1998) Ethylene responses are negatively regulated by a receptor gene family in Arabidopsis thaliana. Cell 94: 261-271
Ingram G C, Waites R (2006) Keeping it together: co-ordinating plant growth. Curr Opin Plant Biol 9: 12-20
Inze D, De Veylder L (2006) Cell cycle regulation in plant development. Annu Rev Genet 40: 77-105
Li Y, Zheng L, Corke F, Smith C, Bevan M W (2008) Control of final seed and organ size by the DA1 gene family in Arabidopsis thaliana. Genes Dev 22: 1331-1336
Mitchum M G, Yamaguchi S, Hanada A, Kuwahara A, Yoshioka Y, Kato T, Tabata S, Kamiya Y, Sun T P (2006) Distinct and overlapping roles of two gibberellin 3-oxidases in Arabidopsis development. Plant J 45: 804-818
Palatnik J F, Allen E, Wu X, Schommer C, Schwab R, Carrington J C, Weigel D (2003) Control of leaf morphogenesis by microRNAs. Nature 425: 257-263
Rieu I, Eriksson S, Powers S J, Gong F, Griffiths J, Woolley L, Benlloch R, Nilsson O, Thomas S G, Hedden P, Phillips A L (2008) Genetic analysis reveals that C19-GA 2-oxidation is a major gibberellin inactivation pathway in Arabidopsis. Plant Cell 20: 2420-2436
Wang H Y, Klatte M, Jakoby M, Baumlein H, Weisshaar B, Bauer P (2007) Iron deficiency-mediated stress regulation of four subgroup Ib BHLH genes in Arabidopsis thaliana. Planta 226: 897-908
White D W (2006) PEAPOD regulates lamina size and curvature in Arabidopsis. Proc Natl Acad Sci USA 103: 13238-13243

1. A method for selecting a suitable plant genotype comprising a phenotype of interest for the introduction of a trait expressing a phenotype related to the phenotype of interest, said method comprising the following steps:
i) providing a genotype collection of immature plants displaying an expected variation of a future phenotype of interest related to the phenotype expressed by the trait wherein the phenotype is only present when the plants are mature,
ii) isolating a tissue from each immature plant in the genotype collection wherein the tissue is determinative for the phenotype,
iii) carrying out a transcriptional profile on each of the tissues,
iv) evaluating the correspondence between a plant phenotype predictor present in the transcriptional profile and the plant phenotype of interest, the correspondences being previously measured by
a) providing a reference genotype collection of immature plants displaying an expected variation of the future phenotype of interest,
b) carrying out steps ii) and iii) in the plants of the reference collection, and
c) determining a plant phenotype predictor associated with the phenotype of interest with a statistical model, and
v) based on said evaluation in step iv) selecting a suitable plant genotype for the introduction of a trait encoding a specific phenotype.

2. A method according to claim 1 wherein said plant phenotype predictor comprises the expression levels of less than 200 genes.

3. A method according to claim 1 wherein said plant phenotype predictor comprises the expression levels of less than 100 genes.

4. The method according to claim 1, wherein the trait is introduced via breeding.

5. The method according to claim 1 wherein the trait is introduced via transformation.

6. The method according to claim 1, wherein the trait is a recombinant trait.

7. The method according to claim 1, wherein the trait is a natural trait.

8. A method for selecting a plant comprising a predicted phenotype of interest, the method comprising the following steps:
i) providing a collection of immature plants displaying a variation of a phenotype of interest wherein said phenotype is only present when the plants are mature,
ii) isolating a tissue from each immature plant in the collection wherein the tissue is determinative for the future phenotype,
iii) carrying out a transcriptional profile on each of the tissues,
iv) evaluating the correspondence between a plant phenotype predictor present in the transcriptional profile and the plant phenotype of interest, the correspondence being previously measured by
a) providing a reference collection of immature plants displaying an expected variation of the future phenotype of interest,
b) carrying out steps ii) and iii) in the plants of the reference collection, and
c) determining a plant phenotype predictor associated with the future phenotype with a statistical model, and
v) based on the evaluation in step iv) selecting a plant comprising a phenotype of interest.

9. A method according to claim 8 wherein said plant phenotype predictor comprises the expression levels of less than 200 genes.

10. A method according to claim 8 wherein said plant phenotype predictor comprises the expression levels of less than 100 genes.

11. The method according to claim 8 wherein the selected plant is a plant genotype selected from a germplasm collection of plants.

12. The method according to claim 8, wherein the plant comprising a phenotype of interest comprises at least one transgenic trait wherein the transgenic trait influences the phenotype of interest.

13. The method according to claim 1, wherein the collection of plants and the reference collection of plants are derived from the same species.

14. The method according to claim 1, wherein the collection of plants and the reference collection of plants are derived from the same genus.

15. The method according to claim 1, wherein the collection of plants and the reference collection of plants are derived from different genera.

16. The method according to claim 8, wherein the collection of plants and the reference collection of plants are derived from the same species.

17. The method according to claim 8, wherein the collection of plants and the reference collection of plants are derived from the same genus.

18. The method according to claim 8, wherein the collection of plants and the reference collection of plants are derived from different genera.
